(19) United States
(12) Patent Application Publication
(10) Pub. No.: US 2009/0205977 A1
(43) Pub. Date: Aug. 20, 2009

(54) Method and system for detecting a target with a specific marker

(76) Inventors: Bor-Yuan Shew, Toufen Township (TW); Shih-Jen Liu, Taichung City (TW); Chih-Hsiang Leng, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/081,630

(22) Filed: Apr. 18, 2008

(30) Foreign Application Priority Data
Feb. 15, 2008 (TW) 97105361

(51) Int. Cl. C12Q 1/00

(52) U.S. Cl. 205/777.5; 204/403.01

(57) ABSTRACT

A method for detecting a target with a specific marker includes: putting a specimen in a reactor, wherein the specimen contains a target with a specific marker, and the reactor has a plurality of biological probes arranged on the bottom; placing the reactor between a first electrode and a second electrode, wherein the area of the first electrode is different from the area of the second electrode; providing a power to the first electrode and the second electrode to generate an electric field for promoting the target to conjugate with the biological probes; and removing the specimen unconjugated with the biological probes. A system for detecting a target with a specific marker is also disclosed. The above-mentioned method and system is appropriate for massive and parallel detection with simpler operation and lower cost.

### BACKGROUND OF THE INVENTION

1. Field of the Invention

The present invention relates to a method and system for detecting a target with a specific marker, particularly to a method and system for improving the isolating efficiency of a target with a specific marker by using a dielectrophoresis technique.

2. Description of the Prior Art

Methods for detecting a target with a specific marker have been commonly applied in the field of biotechnology. For example, there are protein compounds with various structures on the cell surface that have function of providing cell-cell interaction, identification, and signal transduction, etc, and thus play a very important role in the operation of animal immune systems. Therefore, during the process of developing new drugs or vaccines, the concentration of specific cells with particular protein compounds on their surface is an important indicator of evaluating the effect of drugs and vaccines.

A conventional method for detecting a specific cell begins with fluorescent staining cells specifically, and follows by analyzing with a flow cytometer. The flow cytometer allows cells to pass through a particular channel one by one, and then process the real-time counting or sorting with laser-activated fluorescence. However, the flow cytometer is not only expensive but also unable to parallel process massive cell specimens. In addition, due to larger signal/noise ratio of dynamic processing, the specimens requires further cell culture to increase the concentration of specific cells for better testing precision. However, cell culture takes several days, thus the progress of new drug and vaccine development would be severely impacted.

Another conventional method for detecting specific cells captures the specific cells in a specimen with biological probes immobilized on the base plate for following relevant tests. However, this conventional method does not capture specific cells in a specimen efficiently and is not thus applicable for tests containing trace amount of specific cells in the specimen.

To sum up the foregoing description, rapidly, massively, and parallel detecting a target with a specific marker in a specimen with lower cost and less procedures is now urgently needed to target.

### SUMMARY OF THE INVENTION

To solve the above-mentioned problems, one objective of the present invention is to provide a method and system for detecting a target with a specific marker improving the efficiency for biological probes to capture a target with a specific maker by using a dielectrophoresis technique, therefore it is not necessary to proceed cell culture and appropriate for massive and parallel detection.

To achieve the above-mentioned objective, the present invention proposes a method for detecting a target with a specific marker in one embodiment, which includes: putting a specimen in a reactor, wherein the specimen contains a target with a specific marker, and the reactor has a plurality of biological probes immobilized on the bottom; placing the reactor between a first electrode and a second electrode, wherein the size of the first electrode is different from the size of the second electrode; providing a power to the first electrode and the second electrode to generate a weak and non-uniform electric field promoting the target to conjugate with the biological probes; and then removing the specimen unconjugated with the biological probes. The captured cells on the substrate are then imaged and counted.

Other advantages of the present invention will become apparent from the following description taken in conjunction with the accompanying drawings wherein are set forth, by way of illustration and example, certain embodiments of the present invention.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings, wherein:

FIG. 1 is a diagram showing a system for detecting a target with a specific marker according to a preferred embodiment of the present invention

FIG. 2 is a diagram showing the following analysis of a system for detecting a target with a specific marker.

FIG. 3 is a diagram showing reactors arranged as an array according to the present invention.

FIG. 4 is a flow chart showing a method for detecting a target with a specific marker according to a preferred embodiment of the present invention

### DESCRIPTION OF THE PREFERRED EMBODIMENT

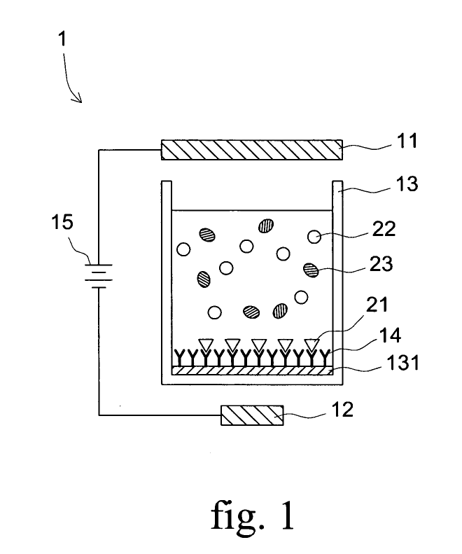

Referring to FIG. 1, a system 1 for detecting a target with a specific marker according to a preferred embodiment of the present invention includes a first electrode 11, a second electrode 12, a reactor 13, a plurality of biological probes 14, and a power supply 15. The size of the first electrode 11 is different from the size of the second electrode 12; i.e. the size of the first electrode 11 may be larger or smaller than the size of the second electrode 12. The reactor 13 is placed between the first electrode 11 and the second electrode 12 for a specimen to be put in. As shown in FIG. 1, the specimen includes a target 21 (e.g. a cell) with a specific marker and particles 22 and 23 without the specific marker.

Following the above description, the biological probes 14 are immobilized on the bottom of the reactor 13. In one embodiment, the biological probes 14 may be proteins (e.g. antibodies and enzymes), antigens, ribonucleic acid or any combination of the above.

It should be noted that a biological film 131 may be arranged on the bottom of the reactor 13, following by immobilizing the biological probes 14 on the biological film 131. For example, the biological film 131 may be PVDF (polyvinylidene difluoride). The power supply 15 is used to provide a power to the first electrode 11 and the second electrode 12 to generate a weak and non-uniform electric field applied to the reactor 13. It should be noted that the power provided by the power supply 15 may be DC or AC. When the electric field is applied to the reactor 13, the target 21 and the particles 22 and 23 in the reactor 13 are driven because of dielectrophoresis effect, and the biological probes 14 may capture the target 21 efficiently.

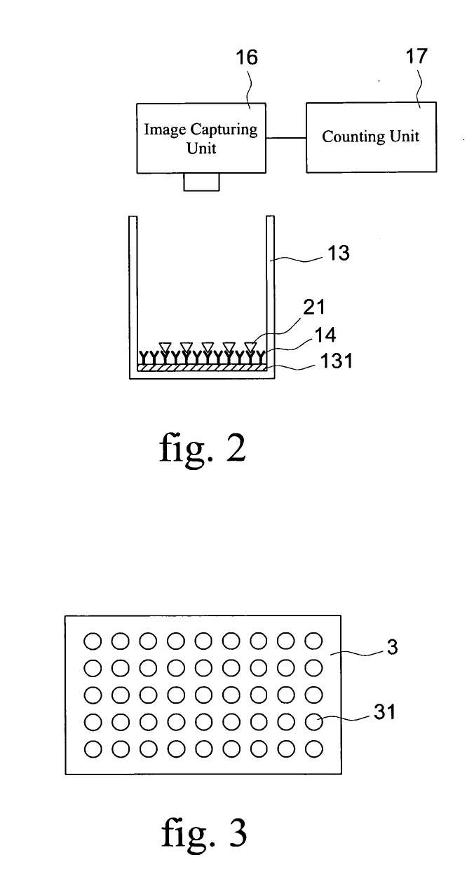

As a result of the target 21 with the specific marker trapped by the biological probes 14 and detained on the bottom of the reactor 13, operators may remove the particles 22 and 23 without the specific marker in the specimen easily and process the following testing procedure of counting or functional analysis using the target 21 with the specific maker. Referring to FIG. 2, the system 1 for detecting a target with a specific marker according to one preferred embodiment of the present invention further includes an image capturing unit 16 used to capture the image of the bottom of the reactor 13 for further analysis. For example, the system 1 of the present invention further includes a counting unit 17 electrically connected to the image capturing unit 16 and used to count the number of the target based on the captured image.

In one embodiment, there are a plurality of the reactors 13 and the reactors 13 are arranged as an array. As shown in FIG. 3, a plurality of notches 31 are formed on a base plate 3, and each of the notches 31 may function as a reactor. In addition, the first electrodes 11 and second electrodes 12 may be arranged as an array (not shown) in correspondence with the notches 31, therefore massive and parallel detection may be processed.

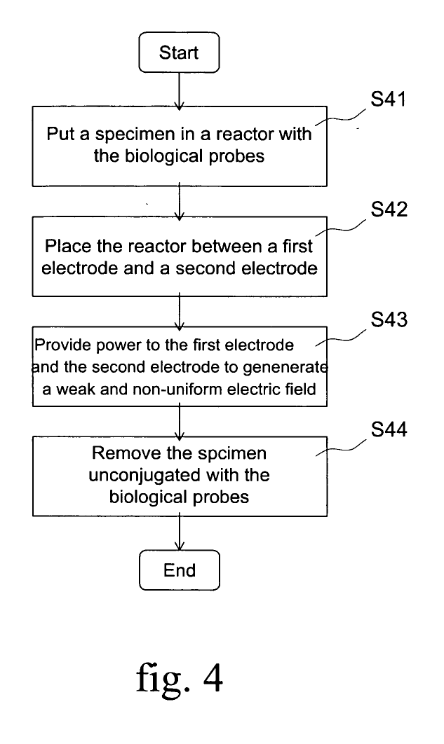

Referring to FIG. 1 and FIG. 4, a method for detecting a target with a specific marker according to one embodiment of the present invention is illustrated. First of all, put a specimen in the reactor 13 (S41), wherein the specimen contains the target 21 with the specific marker and the particles 22 and 23 without the specific marker, and the reactor 13 has a plurality of biological probes 14 immobilized on the bottom; following by placing the reactor 13 between the first electrode 11 and the second electrode 12 (S42), wherein the size of the first electrode 11 is different from the size of the second electrode 12.

Following the above description, provide a power to the first electrode 11 and the second electrode 12 to generate a weak and non-uniform electric field (S43). Due to the different size of the first electrode 11 and the second electrode 12, the electric field between two electrodes is not uniform. The target 21 and particles 22 and 23 are driven by the dielectrophoresis effect, therefore the target 21 is promoted to conjugate with the biological probes 14. In one embodiment, the intensity of the electric field may be 10² to 10⁵ V/m; the temperature of the reactor may be 1° C. to 50° C.; the duration of the electric field may be 2 to 120 minutes. At last, remove the specimen unconjugated with the biological probes 14 (S44).

As a result of the target 21 with the specific marker trapped by the biological probes 14 and detained on the bottom of the reactor 13, operators may remove the particles 22 and 23 without the specific marker in the specimen easily and process the following testing procedure of counting or functional analysis using the target 21 with the specific maker. For example, the target 21 detained on the bottom of the reactor 13 may be stained or fluorescent stained for observation, counted by a image analyzing software, or analyzed for its function, and so on.

It should be noted that operators may process the specimen with stain or fluorescent stain which may be specific or non-specific before processing the method for detecting a target with a specific marker of the present invention. In addition, operators may conjugate the target with a ligand, following by processing the method for detecting a target with a specific marker of the present invention; therefore the biological probes may conjugate with the ligand, or the target is easier to be conjugated with the biological probes.

To sum up the above description, the method and system for detecting a target with a specific marker of the present invention captures the target with the specific marker by using the biological probes and improves the efficiency for biological probes to capture a target with a specific maker by using a dielectrophoresis technique; therefore it is not necessary to proceed cell culture. In addition, the system for detecting a target with a specific marker of the present invention may be arranged as an array therefore is appropriate for rapid, massive and parallel detection with lower cost.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A method for detecting a target with a specific marker comprising:
putting a specimen in a reactor, wherein the specimen contains a target with a specific marker, and the reactor has a plurality of biological probes immobilized on the bottom;
placing the reactor between a first electrode and a second electrode, wherein the size of the first electrode is different from the size of the second electrode;
providing a power to the first electrode and the second electrode to generate a weak and non-uniform electric field promoting the target to conjugate with the biological probes; and
removing the specimen unconjugated with the biological probes.

2. The method as claimed in claim 1, further comprising:
conjugating the specimen with a ligand.

3. The method as claimed in claim 1, wherein the specimen is stained or fluorescent stained.

4. The method as claimed in claim 1, wherein the target is stained or fluorescent stained.

5. The method as claimed in claim 1, further comprising:
counting the number of the target.

6. The method as claimed in claim 5, wherein the counting the number of the target is implemented by an image analyzing software.

7. The method as claimed in claim 1, further comprising:
analyzing the function of the target.

8. The method as claimed in claim 1, wherein the biological probes are proteins.

9. The method as claimed in claim 1, wherein the biological probes are antigens, antibodies, enzymes, ribonucleic acid, or any combination of the above.

10. The method as claimed in claim 1, wherein the size of the first electrode is larger or smaller than the size of the second electrode.

11. The method as claimed in claim 1, wherein the power is DC or AC.

12. The method as claimed in claim 1, wherein the intensity of the electric field is 10² to 10⁵ V/m.

13. The method as claimed in claim 1, wherein the temperature of the reactor is 1° C. to 50° C.

14. The method as claimed in claim 1, wherein the duration of the electric field is 2 to 120 minutes.

15. The method as claimed in claim 1, wherein a plurality of reactors are contained and arranged as an array.

16. The method as claimed in claim 1, wherein a biological film is arranged on the bottom of the reactor, and the biological probes are immobilized on the biological film.

17. The method as claimed in claim 16, wherein the biological film is PVDF (polyvinylidene difluoride).

18. The method as claimed in claim 1, wherein the target is a cell.

19. A system for detecting a target with a specific marker comprising:
a first electrode;
a second electrode, wherein the size of the first electrode is different from the size of the second electrode;
a reactor placed between the first electrode and the second electrode for a specimen to be put in, wherein the specimen contains a target with a specific marker;
a plurality of biological probes immobilized on the bottom of the reactor; and
a power supply providing power to the first electrode and the second electrode to generate a weak and non-uniform electric field promoting the target to conjugate with the biological probes.

20. The system as claimed in claim 19 further comprising:
an image capturing unit used to capture an image of the bottom of the reactor; and
a counting unit electrically connected to the image capturing unit and used to count the targets based on the image.
